(19) United States
(12) Patent Application Publication
(10) Pub. No.: US 2014/0051875 A1
(43) Pub. Date: Feb. 20, 2014

(54) CRYSTALLINE FORM OF MAXACALCITOL

(71) Applicant: FORMOSA LABORATORIES INC., TAOYUAN (TW)

(72) Inventors: CHING-PENG WEI, TAOYUAN (TW); PEI-CHEN HUANG, TAOYUAN (TW)

(73) Assignee: FORMOSA LABORATORIES INC., TAOYUAN (TW)

(21) Appl. No.: 13/968,786

(22) Filed: Aug. 16, 2013

(30) Foreign Application Priority Data
Aug. 17, 2012 (JP) 2012-181178

(51) Int. Cl. C07C 401/00

(52) U.S. Cl. CPC C07C 401/00
USPC 552/653

(57) ABSTRACT

The present invention relates to maxacalcitol hydrate, a new crystalline form of maxacalcitol, with superior technical properties e.g. in the manufacture of crystal suspension formulations, and with superior stability properties.

### FIELD OF THE INVENTION

The present invention relates to maxacalcitol hydrate, a new crystalline form of maxacalcitol, with superior technical properties e.g. in the manufacture of crystal suspension formulations, and with superior stability properties.

### DESCRIPTION OF PRIOR ART

Vitamin D has been long known for its role in calcium and phosphate homeostasis through its actions on the intestine, kidney, bone and parathyroid glands. These actions are mediated by the activated, hormonal form, 1α,25-dihydroxyvitamin D₃ [1α,25(OH)₂D₃], and the vitamin D receptor (VDR) (Molecular Aspects of Medicine 2008, 29(6):433-52). VDR is a member of the steroid/thyroid hormone superfamily, and contains a highly conserved N-terminal DNA binding domain and a less conserved C-terminal ligand binding domain.

The central role of Vitamin D3 in calcium metabolism, cell proliferation, and cell differentiation has made it an attractive candidate for the treatment of a variety of diseases, including cancer, osteoporosis, hyperparathyroidism, and psoriasis. Unfortunately, the high potency of 1α,25(OH)₂D₃ to increase serum calcium and phosphate precludes its therapeutic application in most cases. In response to this limitation, vitamin D analogs have been developed with greater selectivity, which allows more effective intervention with fewer toxic side effects (Molecular Aspects of Medicine 2008;29(6):433-52).

Vitamin D and its derivatives have important physiological functions. The synthetic methods for vitamin D derivatives described in U.S. Pat. No. 4,891,364. And several of these analogs have been approved for use in patients, including calcipotriol (Dovonex®; Leo Pharmaceuticals, Copenhagen, Denmark) and calcipotriene (Daivonex®) for the treatment of psoriasis (U.S. Pat. Nos. 5,292,727 and 4,866,048, respectively), calcitol (1α,25-dihydroxy vitamin D) for the treatment of hyperthyroidism (U.S. Pat. No. 4,308,264), paracalcitol (Zemplar®; Abbott Laboratories, Abbott Park, Ill.) for the treatment of hyperthyroidism (U.S. Patent No. 5,246,925), doxercalciferol (Hectorol®; Bone Care Int, Madison, Wis.) for reduction of elevated parathyroid hormone levels (U.S. Pat. No. 4,555,364), Maxacalcitol (Oxarol®; Chugai Pharmaceuticals, Tokyo, Japan) used as the antihyperparathyroidism and antipsoriatic drug with low calcemic activity (Organic Process Research & Development 2005, 9, 278-287) and alfacalcidol used in regulation of the calcium balance and the bone metabolism (Kidney Int 1990, 38, S22-S27; Nephrol Dial Transplant 2002, 17, 2132-2137; Kidney Int. 1999, 55(3):821-32; Endocrinology 1993, 133, 2724-2728; Curr Opin Investig Drugs. 2004 Sep;5(9):947-51). Novel vitamin D derivatives have been developed to retain effectiveness in the treatment of specific diseases while reducing associated side effects.

Maxacalcitol is a so-called “non-calcemic” vitamin D analog with accentuated differentiation-inducing/antiproliferative properties and reduced ability to cause hypercalcemia. Chemically, maxacalcitol is (+)-(5Z,7E,20S)-20-(3-Hydroxy-3-methylbutoxy)-9,10-secopregna-5,7,10( 19)-triene-1α,3β-diol, also referred to as 22-Oxa-1α,25-(OH)₂ D₃, 22-oxacalcitrol or oxacalcitriol (OTC). Maxacalcitol is the 22-oxa-analogue of 1α,25-dihydroxyvitamin D₃, which contains an oxygen atom in place of carbon 22 of the side chain. Maxacalcitol has been widely used as an antipsoriatic agent and has received a high evaluation from medical experts. In Japan, maxacalcitol is available under the brand name Oxarol® and has been widely used in patients with keratosis including psoriasis vulgaris, remarkably improving the symptoms.

Maxacalcitol, its compound and synthetic methods are mentioned in EP 0184112 A2, WO 2001096293 A and JP 2908566 B2.

The physical property of maxacalcitol can be found in the JP interview form for ointment. The reported melting range of maxacalcitol is 109.8° C. (start melting) to 115.1° C. (all melts). The stability of maxacalcitol is also reported as stable for 36 months in an amber vial under inert gas environment at −80° C., stable for 6 months in an amber vial under inert gas environment at −20° C., and decomposed after 4 weeks storage in an amber vial under inert gas environment at 25° C.

Maxacalcitol with the physical property described above is the anhydrous form, which can be characterized by thermogravimetric analysis (TGA) (see FIG. 1), X-ray powder diffraction (XRD) (see FIG. 2), and differential scanning calorimetry (DSC) (see FIG. 3).

According to the JP interview form, the anhydrous form of maxacalcitol shows a considerable degree of decomposition at 25° C. There is still a need for a more stable form of maxacalcitol.

### BRIEF DESCRIPTION OF THE DRAWINGS

FIG. 1 illustrates the thermogravimetric analysis (TGA) of maxacalcitol anhydrous form. The weight loss of 0.18% at 105° C. for 60 minutes indicates the anhydrous nature.

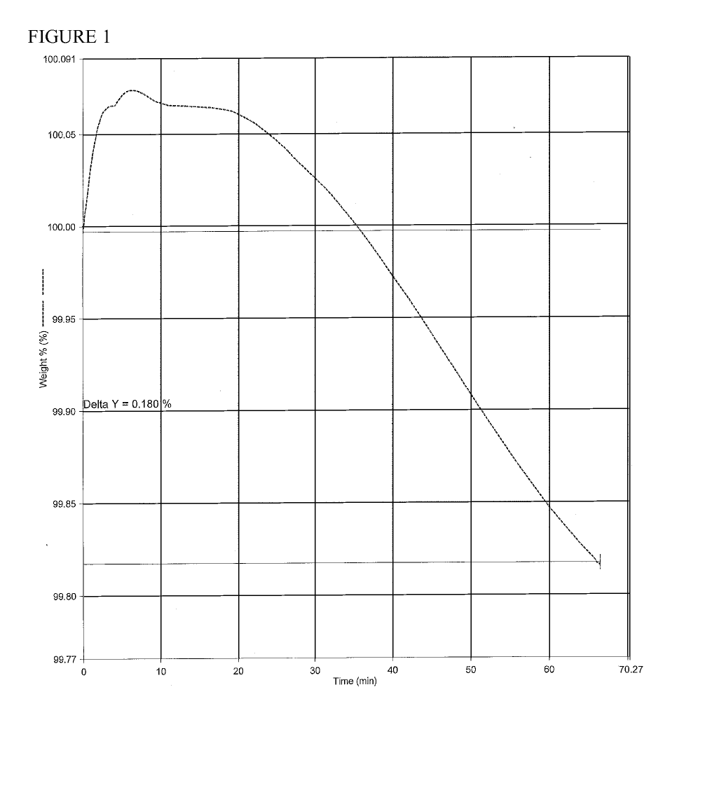

FIG. 2 illustrates the X-ray powder diffraction (XRD) of maxacalcitol anhydrous form

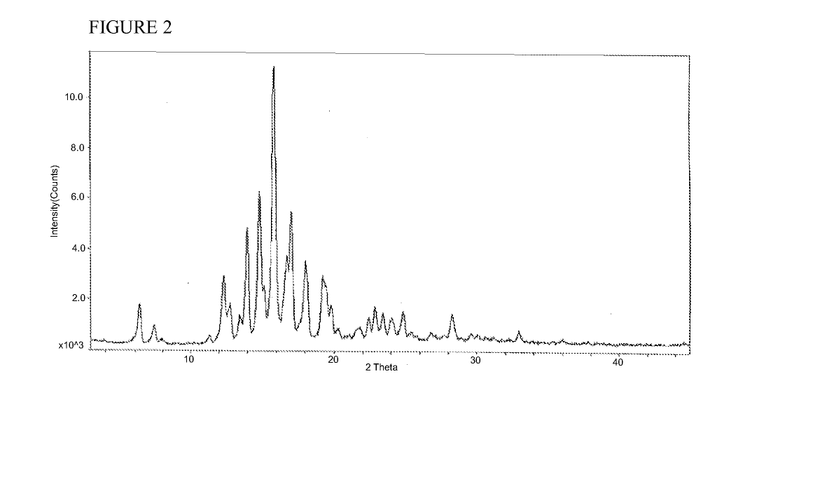

FIG. 3 illustrates the differential scanning calorimetry (DSC) graph of maxacalcitol anhydrous form. The melting range is in compliance with the JP interview form.

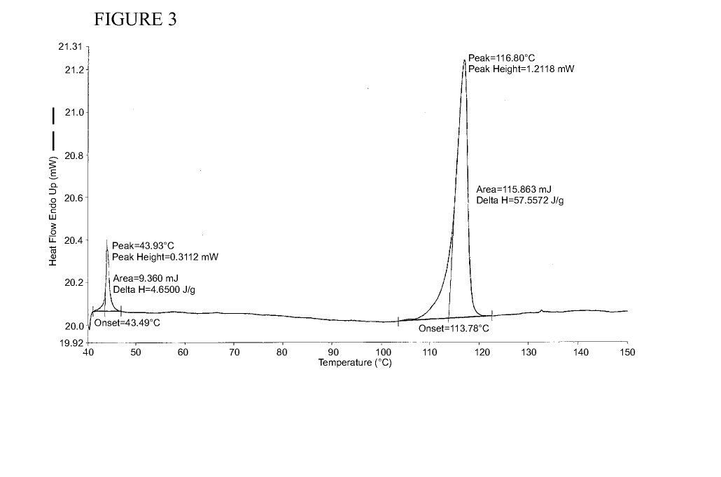

FIG. 4 illustrates the thermogravimetric analysis (TGA) of maxacalcitol hydrate. The weight loss of 4.48% at 120° C. for 240 minutes indicates the monohydrate nature.

FIG. 5 illustrates the X-ray powder diffraction (XRD) of maxacalcitol hydrate.

FIG. 6 illustrates the differential scanning calorimetry (DSC) graph of maxacalcitol hydrate. The melting point at about 86° C. is clearly different from the melting point of the anhydrous form.

### SUMMARY OF THE INVENTION

The present invention provides a cystalline form of maxacalcitol, which is a maxacalcitol hydrate.

The present invention also provides a process for preparing a crystalline form of maxacalcitol hydrate.

### DETAILED DESCRIPTION OF THE INVENTION

The present invention discovered that maxacalcitol can exist in at least two crystalline forms. One is the anhydrous form, which is the only form reported in the literature to date. The present invention provides a new crystalline form of maxacalcitol, maxacalcitol hydrate.

Thermogravimetric analysis (TGA), water content analysis (Karl-Fischer method), X-ray powder diffraction (XRD), and differential scanning calorimetry (DSC) are used to characterize maxacalcitol hydrate.

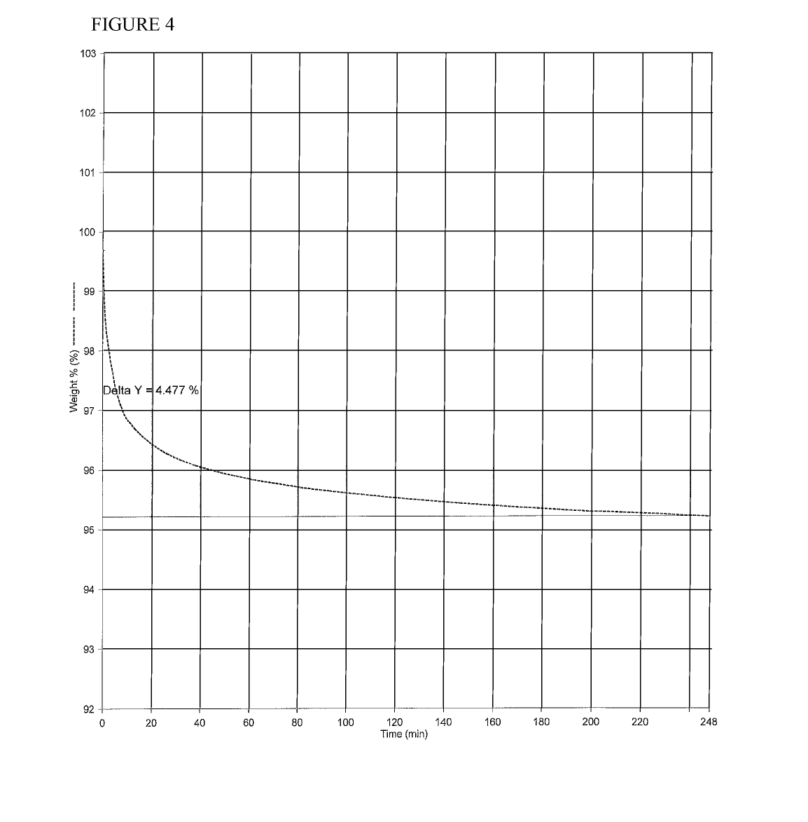

The amount of solvent (including water) in the crystal structure of maxacalcitol hydrate is measured by thermogravimetric analysis (TGA) and the result as shown in FIG. 4. The maxacalcitol hydrate is characterized by a weight loss of about 4.5% at 120° C. for 240 minutes as measured by thermogravimetric analysis (TGA).

The amount of water in the crystal structure of maxacalcitol hydrate is measured by Karl-Fischer (KF) method and the result as shown in Table 1. The maxacalcitol hydrate is characterized by a water content of about 4.2% by weight as measured by Karl-Fischer method. The water content of 4.16% confirms the monohydrate structure.

TABLE 1

| sample size | 0.0 | g |
|---|---|---|
| KFR volume | 0.002 | ml |
| titer | 5.2225 | mg/ml |
| drift auto | 11.4 | μl/min |
| (−d) time | 0:17 | |
| water: division by zero | | |
| sample size | 0.10024 | g |
| KFR volume | 0.798 | ml |
| titer | 5.2225 | mg/ml |
| drift auto | 23.5 | μl/min |

| | |
|---|---|
| (−d) time | 0:33 |
| water | 4.16% |

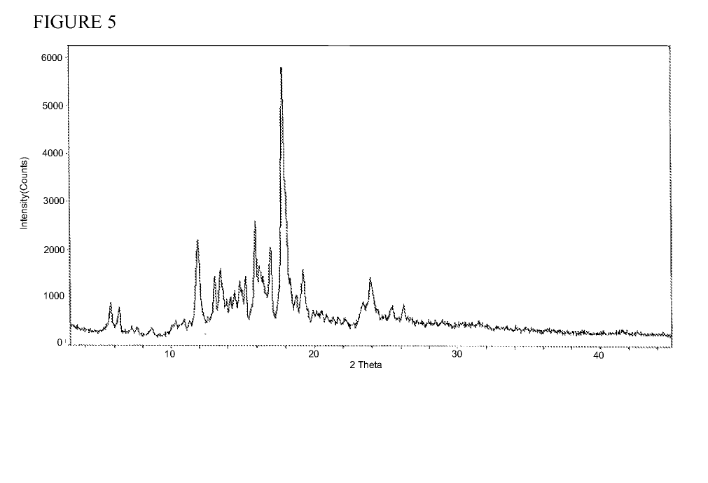

The maxacalcitol hydrate is characterized by a X-ray powder diffraction (XRD) pattern comprising distinctive peaks at 2 theta values of approximately 5.8, 6.3, 12.0, 13.1, 13.5, 13.9, 14.2, 14.5, 14.9, 15.3, 16.0, 16.2, 17.0, 17.9, 18.3, 19.3, 23.5, 24.0, 24.3, 25.4 and 26.2 degree±0.2 degrees 2 theta (FIG. 5).

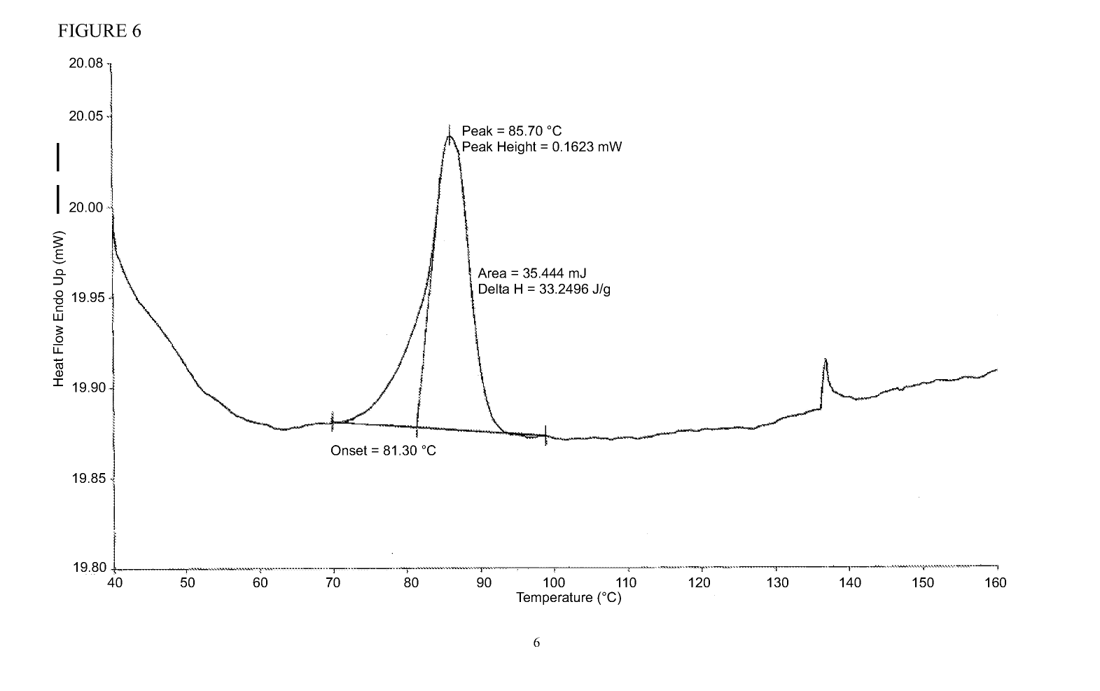

A differential scanning calorimetry (DSC) spectrum of maxacalcitol hydrate as shown in FIG. 6. The maxacalcitol hydrate is characterized by a melting point of about 86° C. as measured by differential scanning calorimetry (DSC) spectrum.

The maxacalcitol hydrate is more stable to storage than anhydrous form, showing no degradation under an inert gas atmosphere at 25° C. for at least 32 days. In a preferred embodiment, the maxacalcitol hydrate is stored in an amber vial.

The present invention also provides a process for preparing a crystalline form of maxacalcitol hydrate comprising:
(a) dissolving a crystalline or non-crystalline maxacalcitol in a polar organic solvent to form a first solution;
(b) combining the first solution with water to form a second solution;
(c) cooling the second solution to form a crystalline precipitate; and
(d) isolating the crystalline precipitate from the second solution to obtain the crystalline form of maxacalcitol hydrate.

In the process of the present invention, the maxacalcitol hydrate isolated from step (d) is a white crystalline powder.

In a preferred embodiment, the polar organic solvent is selected from the group consisting of acetone, acetonitrile, methylformate, methanol, or mixture thereof. In a more preferred embodiment, the polar organic solvent is acetone.

In a preferred embodiment, isolating in step (d) is carried out by filtration, for example by either gravity or suction.

### EXAMPLES

The examples below are non-limiting and are merely representative of various aspects and features of the present invention.

### Example 1

### Preparation of Maxacalcitol Hydrate

Crude maxacalcitol (18.48 g) was dissolved in acetone (87 mL) followed by the addition of water (104.4 mL). The resulting solution was stirred at room temperature for about an hour, cooled to about 8° C., and then kept at the temperature for at least 4 hours. The crystals formed were filtered and dried under vacuum overnight at room temperature to give maxacalcitol hydrate (10.12 g).

### Example 2

### Preparation of Maxacalcitol Hydrate

Crude maxacalcitol (1 g) was dissolved in a mixed solvent of methyl formate (4 mL) followed by the addition of water (0.1 mL). The resulting solution was stirred and cooled to about 8° C., and then kept at the temperature for at least 4 hours. The crystals formed were filtered and dried under vacuum overnight at room temperature to give maxacalcitol hydrate (0.4 g).

### Example 3

### Preparation of Maxacalcitol Hydrate

Crude maxacalcitol (100 mg) was dissolved in acetonitrile (2 mL) followed by the addition of water (3.8 mL). The resulting solution was stirred and cooled to about 8° C., and then kept at the temperature for at least 4 hours. The crystals formed were filtered and dried under vacuum overnight at room temperature to give maxacalcitol hydrate (35 mg).

### Example 4

### Preparation of Maxacalcitol Hydrate

Crude maxacalcitol (100 mg) was dissolved in methanol (0.5 mL) followed by the addition of water (0.5 mL). The resulting solution was stirred and cooled to about 0° C., and then kept at the temperature for at least 4 hours. The crystals formed were filtered and dried under vacuum overnight at room temperature to give maxacalcitol hydrate (45 mg).

### Example 5

### Preparation of Maxacalcitol Anhydrous Form

Maxacalcitol (1 g) was dissolved in butyl acetate (3 mL). The solution was stirred and cooled at 4-6° C. overnight. The crystals formed were filtered and dried under vacuum overnight at room temperature to give maxacalcitol anhydrous form (0.56 g).

What is claimed is:

1. A crystalline form of maxacalcitol, which is maxacalcitol hydrate.

2. The crystalline form of claim 1, characterized by a X-ray powder diffraction (XRD) pattern comprising distinctive peaks at 2 theta values of approximately 5.8, 6.3, 12.0, 13.1, 13.5, 13.9, 14.2, 14.5, 14.9, 15.3, 16.0, 16.2, 17.0, 17.9, 18.3, 19.3, 23.5, 24.0, 24.3, 25.4 and 26.2 degree±0.2 degrees 2 theta.

3. The crystalline form of claim 1, characterized by a weight loss of about 4.5% at 120° C. for 240 minutes as measured by thermogravimetric analysis (TGA).

4. The crystalline form of claim 1, characterized by a water content of about 4.2% by weight as measured by Karl-Fischer method.

5. The crystalline form of claim 1, characterized by a melting point of about 86° C. as measured by differential scanning calorimetry (DSC) spectrum.

6. The crystalline form of claim 1, which is more stable to storage than anhydrous form, showing no degradation under an inert gas atmosphere at 25° C. for at least 32 days.

7. A process for preparing a crystalline form of maxacalcitol hydrate comprising:
(a) dissolving a crystalline or non-crystalline maxacalcitol in a polar organic solvent to form a first solution;
(b) combining the first solution with water to form a second solution;
(c) cooling the second solution to form a crystalline precipitate; and
(d) isolating the crystalline precipitate from the second solution to obtain the crystalline form of maxacalcitol hydrate.

8. The process of claim 7, wherein the crystalline form of maxacalcitol hydrate is characterized by a powder X-ray diffraction (XRD) pattern comprising distinctive peaks at 2 theta values of approximately 5.8, 6.3, 12.0, 13.1, 13.5, 13.9, 14.2, 14.5, 14.9, 15.3, 16.0, 16.2, 17.0, 17.9, 18.3, 19.3, 23.5, 24.0, 24.3, 25.4 and 26.2 degree±0.2 degrees 2 theta.

9. The process of claim 7, wherein the polar organic solvent is acetone, acetonitrile, methyl formate, methanol or mixture thereof.

10. The process of claim 7, wherein the polar organic solvent is acetone.
